(19) United States
(12) Patent Application Publication
(10) Pub. No.: US 2005/0064564 A1
(43) Pub. Date: Mar. 24, 2005

(54) Method and device for producing an aqueous acrylamide solutions using a biocatalysts

(76) Inventors: Olaf I. Petersen, Meerbusch (DE); Burkhard Theis, Moers (DE); Michael Colberg, Willich (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/475,965

(22) PCT Filed: Apr. 25, 2002
(86) PCT No.: PCT/EP02/04565

(30) Foreign Application Priority Data
Apr. 26, 2001 (DE) 10120555.4

(51) Int. Cl. C12P 13/02

(52) U.S. Cl. 435/129; 564/128

(57) ABSTRACT

The invention relates to a method and a device for producing an aqueous acrylamide solution by the hydration of acrylnitrile in an aqueous solution in the presence of a biocatalyst.

The present invention relates to a method and a device for producing an aqueous acrylamide solution by hydrating acrylonitrile in an aqueous solution in the presence of a biocatalyst.

The conversion of acrylonitrile into acrylamide in the presence of a suitable biocatalyst in water has been known for many years and is described, for example, in DE 30 17 005 C2, whereby in this method the biocatalyst is immobilised. DE 44 80 132 C2 and EP 0 188 316 B1 describe special biocatalysts for the conversion of acrylonitrile into acrylamide. U.S. Pat. No. 5,334,519 teaches the hydration of acrylonitrile to form acrylamide in the presence of biocatalysts and cobalt ions. All these teachings have the drawback that undesirable by-products are produced.

Therefore, it is the object of this invention to provide a method which is as environmentally friendly as possible in which by-products are minimised.

According to the invention, the object is achieved by a method for producing an aqueous acrylamide solution by the hydration of acrylonitrile in an aqueous solution in the presence of a biocatalyst in which the biocatalyst is separated from the aqueous acrylamide solution within ≦2 hours, preferably within ≦1 hour of the end of the reaction.

At the start of the reaction, water and the biocatalyst are placed in the reactor and heated to a temperature of 15 to 25° C., preferably 16 to 20° C. When the temperature is reached, the acrylonitrile is added to the reactor and conversion to acrylamide commences. Preferably, the entire conversion takes places isothermally whereby cooling is necessary during the entire conversion in order to draw off the reaction heat. With regard to the cooling of the reaction mixture, reference is made to the parallel application with the internal file number ST0031, which is introduced here as a reference and hence should be considered to be part of the disclosure. At the start of reaction, the concentration of the biomass is preferably 0.03-2.5 g/l, particularly preferably 0.05-1 g/l and the pH value is preferably 6.0-8.0, particularly preferably 6.8-7.5.

When the addition of the acrylonitrile is completed, a secondary reaction of preferably 4 to 20 minutes, particularly preferably 5 to 10 minutes, is required to convert the acrylonitrile as completely as possible.

The reaction is finished for the purposes of the invention when the residual content of acrylonitrile in the aqueous acrylamide solution is less than 10 ppm, preferably less than 5 ppm.

According to the invention, after the end of reaction, the biocatalyst is separated from the aqueous acrylamide solution within ≦2 hours, preferably within ≦1 hour.

Preferably, the biocatalyst is separated by means of a tubular centrifuge, such as that described, for example, by Dr.-Ing. Heinz Hemfort in “Separators”, Technical and Scientific Documentation. The documentation may be obtained from the company GEA Westfalia Separator AG, Wemer-Habig-Strasse 1, D-59302 Oelde and is hereby introduced as a reference and hence should be considered to be part of the disclosure.

Also preferably, the biocatalyst is separated with an at least partially continuous self-draining centrifuge. Particularly preferably, this centrifuge is an annular gap centrifuge such as that described, for example, by Dr.-Ing. Heinz Hemfort in “Separators”, Technical and Scientific Documentation.

In a preferred embodiment of the invention, the clear discharge from the centrifuge is monitored using optical means. This optical means is preferably a light barrier set to the desired degree of turbidity of the acrylamide in the clear discharge. The light barrier is incorporated in a discharge valve in the centrifuge and transmits light through the discharged aqueous acrylamide solution. The light barrier comprises a light source and a receiver. The light intensity of the light source is preferably set so that the light beam attenuated by absorption in the transilluminated aqueous acrylamide solution arrives at the receiver with a residual intensity sufficient to enable the receiver to indicate that the separation of the biocatalyst is adequate. If the onset of turbidity caused by the biocatalyst causes the light absorption to be greater, the light intensity is reduced and the receiver emits a signal indicating that the separation of the biocatalyst is no longer adequate. This signal is preferably used to control the centrifuges. Preferably, this signal is used to regulate the draining or cleaning intervals of the centrifuges.

Advantageously, the biocatalyst is flocculated before the separation. The flocculation may be performed in the same reactor in which the acrylonitrile is converted into acrylamide. However, preferably, the flocculation is performed in a separate flocculation vessel. The flocculation may be performed with any suitable flocculation agent. However, advantageously, the flocculation is performed with aluminium sulphate and/or with an anionic polymer. Suitable anionic polymers are, for example, the applicant's products Praestol® 2510 or Praestol® 2530.

Preferably, the flocculation is performed at a pH value of 6.8 to 8.0, particularly preferably at a pH value of 7.0 to 7.5.

When the biocatalyst, the biomass, has been freed of the aqueous acrylamide solution, the aqueous acrylamide solution is preferably set to a pH value of from 4.5 to 7.0, particularly preferably from 5.5 to 6.5.

In a preferred embodiment of the invention, the biocatalyst is at least to a large extent freed of acrylamide by at least a single, particularly preferably multiple washing and separation of the washing water. Preferably, the washing is performed with deionised water. Also preferably, the biocatalyst is washed until the acrylamide concentration in the biocatalyst is <10 ppm, particularly preferably <5 ppm.

The washing water loaded with acrylamide is recycled in the process and, for example, placed in the reactor. The biocatalyst is then suspended in this water before the actual conversion of acrylonitrile into acrylamide commences.

After washing, the biocatalyst is preferably sterilised and then disposed of as normal biowaste. Sterilisation is preferably performed by briefly heating the biocatalyst to temperatures of >80° C.

The method according to invention may be performed with any biocatalyst that catalyses the conversion of acrylonitrile into acrylamide. Preferably, however, the biocatalyst is a Rhodococcus rhodochrous deposited under the deposition number 14230 with DSMZ, Deutsche Sammlung von Mikroorganismen und Zellkulturen GmbH (German Collection of Microorganisms and Cell Cultures Ltd), Mascheroder Weg 1b, D-38124 Braunschweig, Germany.

The method according to the invention has the advantage that fewer by-products are produced, the conversion of the acrylonitrile takes place at least almost completely and that an acrylamide solution of up to 50% by weight is achievable. The method according to the invention is simple and inexpensive to perform. The biocatalyst is utilised to the optimum extent and may be disposed of as biowaste. The water used to wash the biocatalyst may be recycled in the process.

The method according to the invention is preferably performed in a device for the production of an aqueous acrylamide solution by the hydration of acrylonitrile in an aqueous solution in the presence of a biocatalyst with a reactor and a tubular centrifuge for separating the biocatalyst from the aqueous acrylamide solution. Therefore, this device is a further subject of this invention. Tubular centrifuges are described, for example, by Dr.-Ing. Heinz Hemfort in “Separators”, Technical and Scientific Documentation.

A further subject of the invention is a device for the production of an aqueous acrylamide solution by the hydration of acrylonitrile in an aqueous solution in the presence of a biocatalyst with a reactor and an at least partially continuous self-draining centrifuge for separating the biocatalyst from the aqueous acrylamide solution.

Preferably, the at least partially continuous self-draining centrifuge is a self-draining annular gap centrifuge or an annular gap plate centrifuge such as that described, for example, by Dr.-Ing. Heinz Hemfort in “Separators”, Technical and Scientific Documentation

Reference is made to the above explanations with reference to the regulation of the centrifuges.

The device according to the invention has the advantage that fewer by-products are produced, the conversion of the acrylonitrile takes place at least almost completely and that an acrylamide solution of up to 50% by weight is achievable. The device according to the invention is simple and inexpensive to operate. The biocatalyst is utilised to the optimum extent and may be disposed of as biowaste.

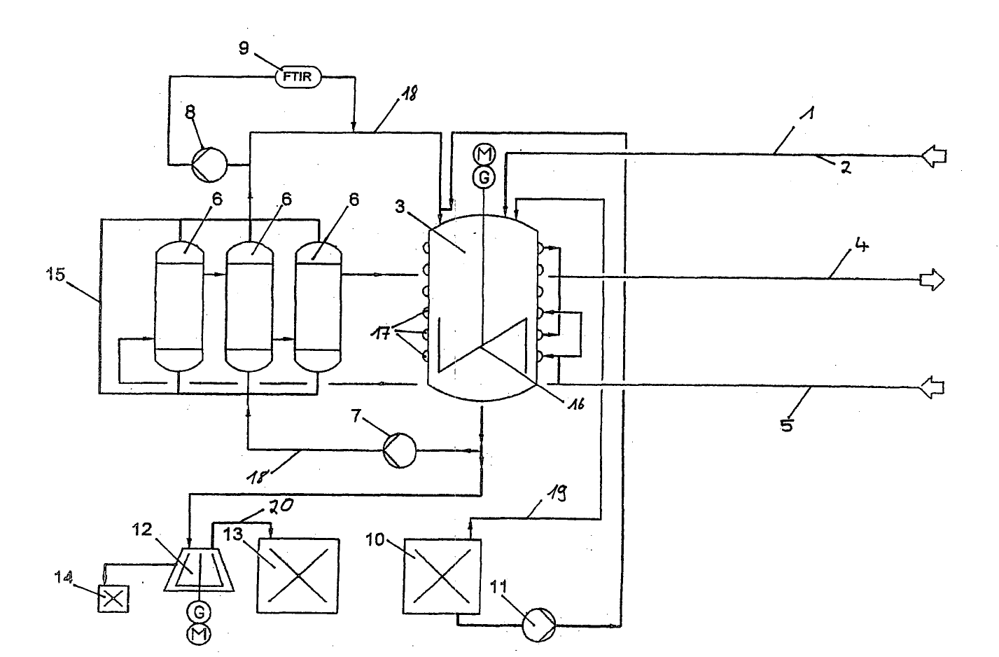

The invention will be further described with reference to FIG. 1. However, these explanations are by way of example only and do not restrict the general concept of the invention.

FIG. 1 is a schematic diagram of the method according to the invention or parts of the device according to the invention. Before the start of the actual conversion of acrylonitrile into acrylamide, deionised water 1 and a suspension 2, containing the biocatalyst, are placed in the reactor 3. The reactor 3 is mixed homogenously with a motor-driven agitator 16. On the exterior of the reactor 3, there are cooling coils 17 which are connected to the cold water inlet 5 and the cold water outlet 4. A person skilled in the art will recognise that these cooling coils can also be used to heat the reactor content to a specific temperature before the start of the actual reaction.

In addition, the reactor 3 comprises a pumping circuit 18 through which a part of the reactor content is circulated by means of the magnetically coupled side channel pump 7. Arranged in the pumping circuit 18 are three shell-and-tube heat exchangers 6 connected in parallel with which the reactor content may be heated or cooled. The heat exchangers 6 are also connected to the cold water inlet or outlet. In addition, the pumping circuit comprises the bypass 15 with which the heat exchanger 6 may be bypassed. The corresponding valves are not shown. The pumping circuit also contains the Fourier transform infrared device (FT-IR device) 9 for the on-line measurement of the acrylonitrile and acrylamide concentration in the circulated flow and hence in the reactor. The sample flow is taken from the pumping circuit 18 and sent by means of the piston-diaphragm pump 8 to the FT-IR device 9 where it is analysed. The analytical data are used to control the method. Shortly before the pumping circuit re-enters the reactor, the acrylonitrile to be converted is added to it from the acrylonitrile receiver 10 by means of the diaphragm-feed pump 11. The acrylonitrile receiver 10 and the reactor 3 are connected to each other by means of a pendulum line 19 at the gas side. The line 19 is opened before the addition of the acrylonitrile commences and closed again when the addition is completed. When the addition of the acrylonitrile is complete, a secondary reaction of preferably 5-20 minutes is required to convert the acrylonitrile at least almost completely. The reaction is considered to be completed when the acrylamide concentration in the biocatalyst is <10 ppm.

When the reaction has finished, the suspension is pumped into a separate vessel (not shown) and the biocatalyst flocculated at a pH value of 7.0 to 7.5 with aluminium sulphate. Then, the biocatalyst is separated from the acrylamide in a partially continuous self-draining annular gap centrifuge 12 made by the company GEA Westfalia Separator AG, Wemer-Habig-Strasse 1, D-59302, Federal Republic of Germany, whereby the separation is completed at least one hour after the end of the reaction. The annular gap centrifuge is controlled by the signal from a light barrier (not shown) located in the line 20. In particular, the signal from the light barrier controls the partially continuous drainage of the centrifuge. The aqueous acrylamide is collected in the receiver 13 and set to a pH value of 5.5 to 6.5. The biocatalyst is collected in the receiver 14 and then washed several times in deionised water and drained in order to free the biocatalyst of acrylamide. The washing water is recycled back in the process via the line 1. The washed biocatalyst is sterilised with steam and disposed of as biowaste.

1. Method for producing an aqueous acrylamide solution by the hydration of acrylonitrile in an aqueous solution in the presence of a biocatalyst, characterised in that the biocatalyst is separated from the aqueous acrylamide solution within ≦2 hours, preferably within ≦1 hour of the end of the reaction.

2. Method according to claim 1, characterised in that the biocatalyst is separated with a tubular centrifuge.

3. Method according to claim 1, characterised in that the biocatalyst is separated with an at least partially continuously operating, self-draining centrifuge.

4. Method according to claim 3, characterised in that the centrifuge is an annular gap centrifuge.

5. Method according to any one of claims 2 to 4, characterised in that the clear discharge from the centrifuge is preferably monitored using an optical means, particularly preferably a light barrier.

6. Method according to claim 5, characterised in that the monitoring is used to control the centrifuges.

7. Method according any one of claims 1 to 6, characterised in that the biocatalyst is flocculated before the separation.

8. Method according to claim 7, characterised in that aluminium sulphate is used as the flocculation agent.

9. Method according to claim 7, characterised in that an anionic polymer is used as the flocculation agent.

10. Method according to any one of claims 7 to 9, characterised in that the flocculation is performed at a pH value of 6.8 to 8.0, preferably 7.0 to 7.5.

11. Method according to any one of claims 1 to 10, characterised in that the aqueous acrylamide solution freed of biocatalyst is set to a pH value of 4.5 to 7.0, preferably 5.5 to 6.5.

12. Method according to any one of claims 1 to 11, characterised in that the separated biocatalyst is freed of acrylamide by at least a single, preferably multiple washing and separation.

13. Method according to claim 12, characterised in that the washing is performed with deionised water.

14. Method according to claim 12 or 13, characterised in that the acrylamide concentration in the biocatalyst is <10 ppm, preferably <5 ppm.

15. Method according to any one of claims 12 to 14, characterised in that the washing water is recycled in the process.

16. Method according to any one of claims 12 to 15, characterised in that the biocatalyst is sterilised after the washing.

17. Method according to any one of claims 1 to 16, characterised in that the biocatalyst is Rhodococcus rhodochrous filed under the deposition number 14230 with DSMZ, Deutsche Sammlung von Mikroorganismen und Zellkulturen GmbH, Maschroder Weg 1b, D-38124 Braunschweig, Germany.

18. Device for the production of an aqueous acrylamide solution by the hydration of acrylonitrile in an aqueous solution in the presence of a biocatalyst with a reactor and a tubular centrifuge for separating the biocatalyst from the aqueous acrylamide solution.

19. Device for the production of an aqueous acrylamide solution by the hydration of acrylonitrile in an aqueous solution in the presence of a biocatalyst with a reactor and a self-draining, at least partially continuously operating centrifuge, to separate the biocatalyst from the aqueous acrylamide solution.

20. Device according to claim 19, characterised in that the centrifuge is an annular gap centrifuge.

21. Device according to any one of claims 18 to 20, characterised in that the clear discharge from the centrifuge is monitored using an optical means.

22. Device according to claim 21, characterised in that a signal is used to control the centrifuge.
